(19) United States
(12) Patent Application Publication
(10) Pub. No.: US 2015/0096569 A1
(43) Pub. Date: Apr. 9, 2015

(54) Pleasurable Protective Undergarment

(71) Applicant: Jamie S. Jones, Birmingham, AL (US)

(72) Inventor: Jamie S. Jones, Birmingham, AL (US)

(21) Appl. No.: 14/048,437

(22) Filed: Oct. 8, 2013

(51) Int. Cl. A41B 9/12; A61F 6/06; A61F 6/04

(52) U.S. Cl. CPC A41B 9/12; A61F 6/04; A61F 6/06
USPC 128/846

(57) ABSTRACT

A garment that is made in the form of underwear, that provides a protective barrier between sex partners. The garment provides protection from HPV (human papillomavirus), and genital ulcer diseases such as; genital herpes, syphilis, and chanchroid, while making sex possible. The garment is made of both a breathable and non-breathable fabric that prohibits the transfer of sexually transmitted diseases (listed above) by way of an infected partner's skin. The garment in the most basic form is a breathable fabric in the form of underwear that has an additional layer of latex in the crotch area, and openings for the insertion of a penis. The garment is made differently for males and females to address the differing locations of female/male anatomy.

### BACKGROUND OF THE INVENTION

The following invention adds additional protection to the condom to prevent the spread or transmittal of all STD's, including the “skin to skin” contractible diseases that are not covered by the condom alone. The garment picks up where the condom leaves off by providing coverage for the skin that is most in contact with the infected area.

For protection during sexual acts a condom is most widely encouraged as a way to prevent pregnancy and catching sexually transmitted diseases. Little emphasis is placed on diseases transmitted around the condom through skin on skin contact. The skin on skin infectious disease rate is affected by the lack of items available to prevent transmittal. These diseases sometimes are not immediately noticed, increasing the chance for further spreading, and are painful, embarrassing, and in some cases incurable. This invention can be used in a non-disposable or disposable form by persons in a casual relationship or those in a relationship in which one partner is infected and the other person isn't. This invention prevents uninfected people from having to jeopardize their health in either of these relationships

### DISCRIPTION OF THE PRIOR ART

Prior U.S. Pat. Nos. 5,596,997; 5,535,757; are different from this invention because these inventions seek to include a device to cover the penis, while not covering the thigh and complete crotch area. The PLEASURABLE PROTECTIVE UNDERGARMENT invention seeks to cover the hips, complete crotch area, and some thigh, and not provide a cover for the penis. The female panty condom is also different from the pleasurable protective undergarment in purpose because the female panty condom's use is aimed at birth control.

U.S. Pat. No. 4,664,104, Anti-herpes modality system (AHMS), is different from the pleasurable protective undergarment because the Anti-herpes modality system has a sheath member to be connected to the garment by way of biasable gripper to fit around a toroidal (toridal) lock secured to the rim of a hole. This invention does not attempt to attach, connect, lock, or secure a sheath, cover, or condom. The AHMS has multiple parts with a more complex design. The pleasurable protective undergarment seeks to prevent the transmittal of diseases with a much simpler garment that is easy to use, more convenient, and comfortable. The Pleasurable protective undergarment does not seek to limit or decide the consumer's choice of prophylaxis.

U.S. Pat. No. 4,840,624, female condom device, has a female condom attached to it, and does not attempt to cover surrounding skin to prevent the transmittal of skin to skin contact diseases. U.S. Pat. No. D527167, Protective briefs for sexually-active persons, seems to have condoms provided in both the vaginal and anal area, which may be uncomfortable for non- anal users. It also is not specified whether the garment is for males or females. This invention differs from the pleasurable protective undergarment because it is completely closed around the clitoral or scrotum area, whereas the pleasurable protective panty can be opened for clitoral or scrotal stimulation. U.S. Pat. No. D523140, Male prophylactic garment does not attempt to prevent the transmission of diseases spread through skin on skin contact. U.S. Pat. No. 5,687,741, Garment for releasable securing condoms, is a bikini shaped panty that has a snap for opening a flap to access the vagina. This invention is different from the pleasurable protective undergarment, because it does not attempt to prevent skin contact of surrounding areas from being infected, has a snap to a flap, attempts to secure the rim of the condom to the panty, attempts for a woman to wear a condom for a prolonged amount of time prior to intercourse, and has a pocket attached for a condom.

Many inventions used during sex are to prevent pregnancy, or the slipping of condoms. The prior inventions with goals of preventing the spread of skin communicable diseases, seek to do so in non-practical ways without regards to comfort for both partners, and appeal, making them more unlikely to be acceptable. Many garments including U.S. Pat. No. D419233, D527167 seem to integrate the penis cover, or prophylaxis, and also have not addressed the ability of manual clitoral or scrotal stimulation, which disengages many consumers. The pleasurable protective undergarment seeks to provide pleasure, appeal, style, comfort, and protection while also being reasonable and unobtrusive. No other garment claims a double layer, 1) a breathable fabric 2) non-breathable fabric (such as latex, nylon, etc.) in the crotch area, or the ability for manual clitoral, scrotal, and anal stimulation.

### BRIEF SUMMARY OF THE INVENTION

This invention is a protective undergarment for sexual contact that prevents skin to skin contact in genital area. The idea is to be completely protected from contracting or spreading STD's, and to make protection acceptable and sexy. The undergarment would come in different colors, styles, and fabrics. The garment will be made for both disposable wear, as condoms are, and non- disposable, for couples and people wishing to be protected all of the time. The undergarment would not include a condom, but will have an area(s) for penile entrance, enclosing around penis to provide complete protection from skin contact in the crotch area. The product will be made for men and women, homosexual and heterosexual. The garment will not have a hole or ring, but an overlapping of fabric in the crotch area that can be opened by pulling both sides of fabric back. The PLEASURABLE PROTECTIVE UNDERGARMENT would have multiple layers in the crotch area for additional protection in the genital zone, which is also susceptible to being infected.

### BRIEF DESCRIPTION OF THE DRAWING

The above and other objects, features, and advantages of the invention will become more apparent from the following description of certain embodiments thereof, when taken in conjunction with the accompanying drawings in which:

DRAWING. 1 is a perspective view of a woman's garment generally from the front, showing a person wearing an undergarment in accordance with a preferred embodiment of the invention;

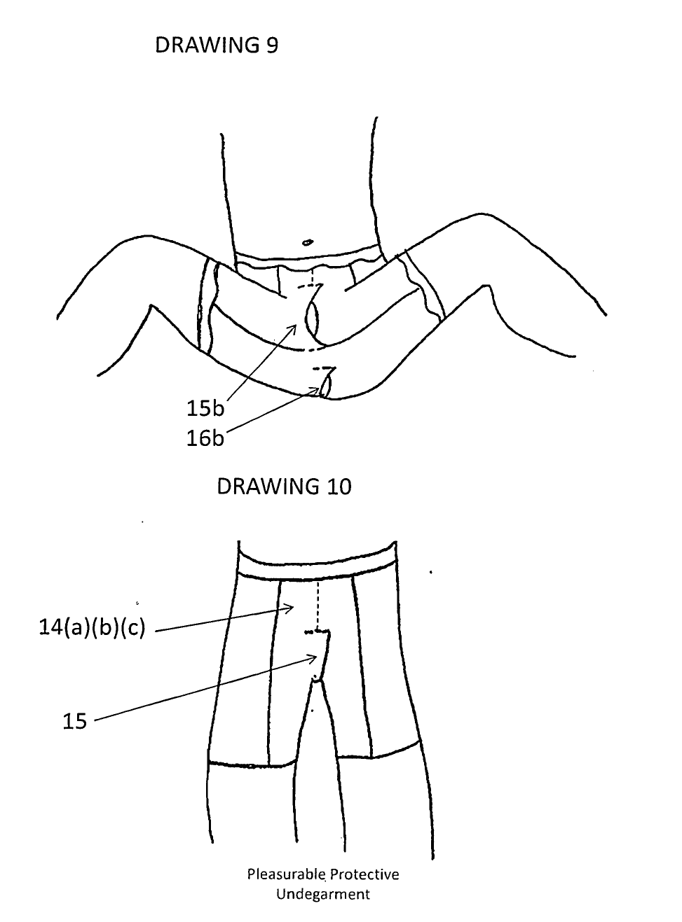

DRAWING. 2 is an exploded view of FIG. 14, the layered zone of the garment

DRAWING. 3 is a perspective view of a woman's garment, generally from the rear, showing the person wearing the undergarment of DRAWING. 1

DRAWING. 4 is the exploded view of the first layer of three layers which FIG. 14 is composed of; the first layer of FIG. 14 is that of a breathable fabric

DRAWING. 5 is the exploded view of FIG. 14(b) the second of three layers which FIG. 14 is composed of; the second layer of FIG. 14 is that of a non- breathable fabric such as latex

DRAWING. 6 is the exploded view of FIG. 14(c) the third, and last of three layers which FIG. 14 is composed of; the third layer of FIG. 14 is that of a breathable fabric

DRAWING. 7 is a perspective view, showing a person wearing an undergarment with the vertical elongated opening of the layered genital zone in an open position

DRAWING. 8 is a center view of the undergarment, showing the front genital opening, and an optional opening for the rear, both in closed positions

DRAWING. 9 is a center view of the undergarment, showing the front genital opening, and an optional opening for the rear, both in open positions

DRAWING. 10 is a perspective view of a male's undergarment from the front, showing a male wearing the undergarment in accordance with the preferred embodiment of the invention

DRAWING. 11 is a perspective view of a male's undergarment from the front, with the elongated opening of the layered zone in an open position

DRAWING. 12 is a view of the front of a male garment in an open position

### DETAILED DESCRIPTION OF THE DRAWINGS

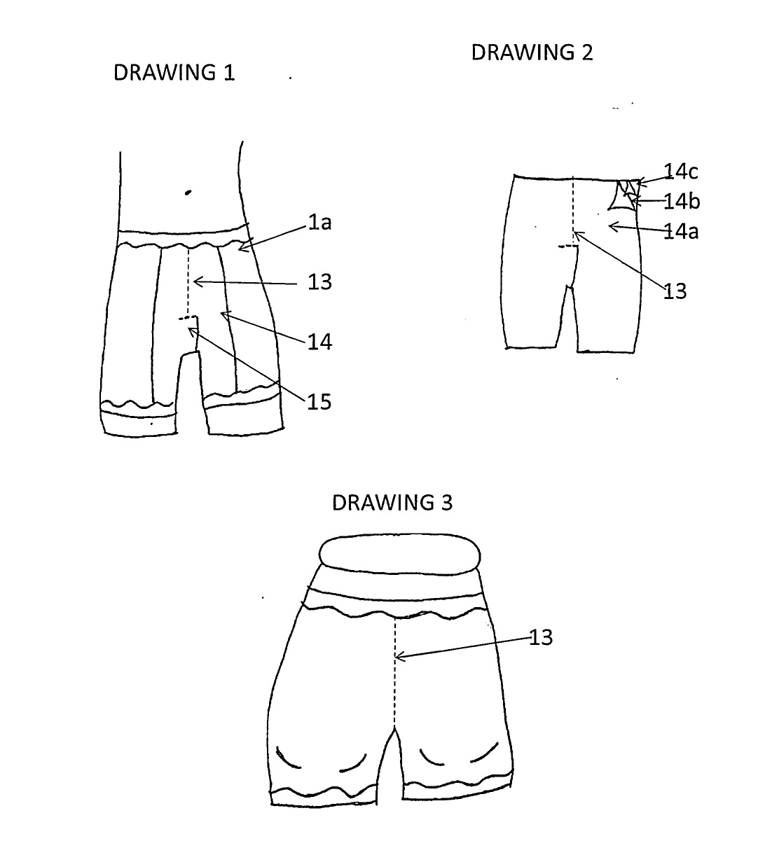

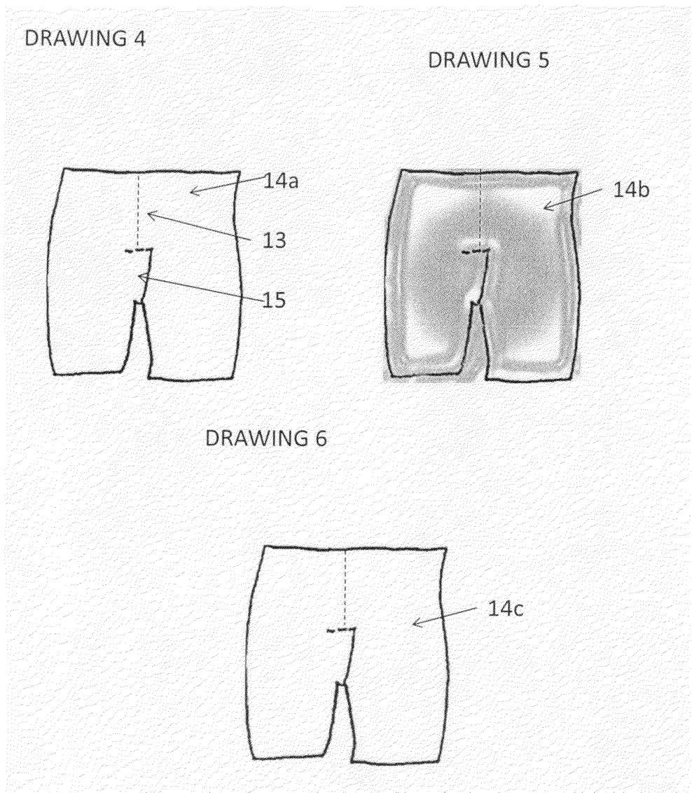

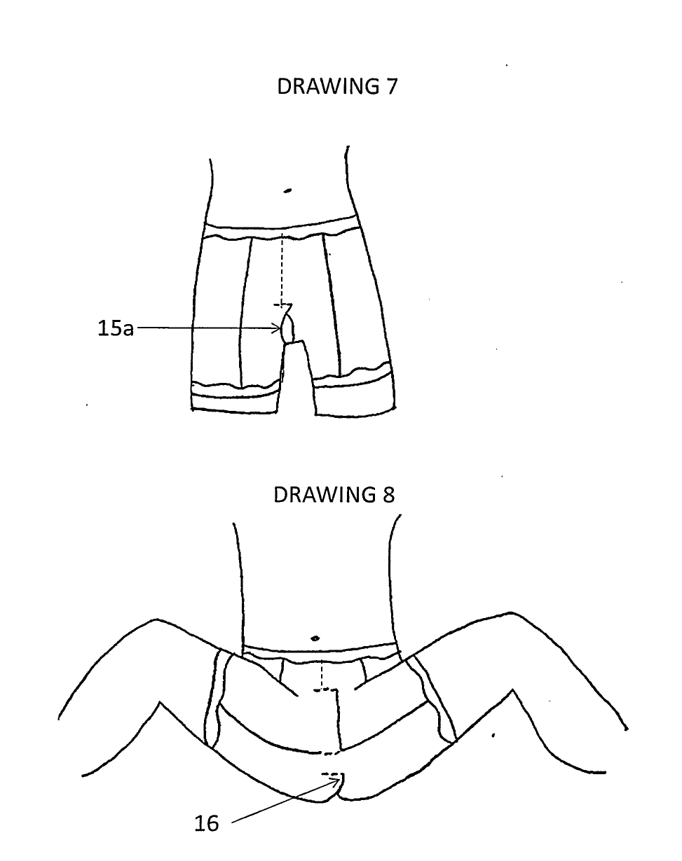

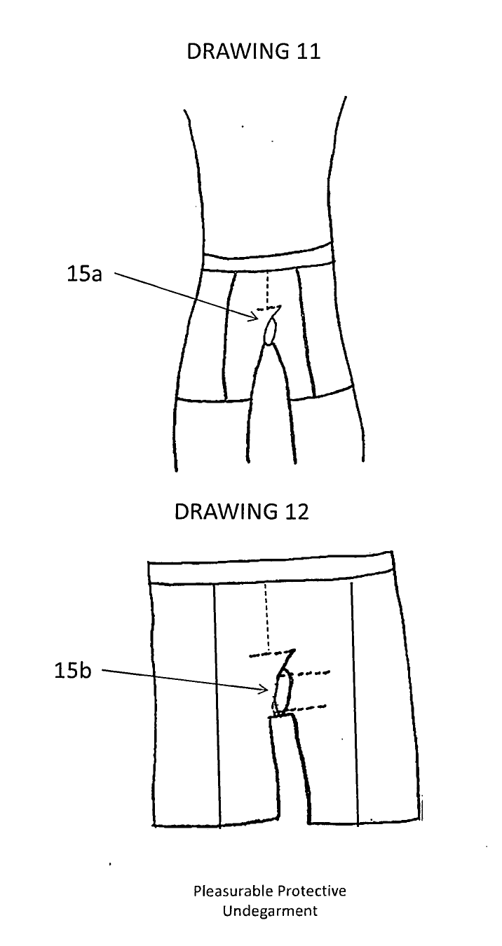

DRAWING 1 is an undergarment in the form of a boy short version of women's underwear. The undergarment, as a cover for the lower abdomen, waist, hips, crotch, and upper to mid-thigh, is made of a breathable fabric throughout the garment. FIG. 1a is the complete women's pleasurable protective undergarment, it includes; FIG. 14, shown in an exploded view in Drawing 2, is the layered zone. FIG. 14, shown in DRAWING. 2 includes 3 parts: 14a, 14b, and 14c. 14a is the outer and first layer of the layered zone of the garment. 14a is made of a breathable fabric in which the whole garment is made of. It has FIG. 15, the vertical elongated opening, in which the seams of the two sides overlap each other by 1 to 2 inches down the center seam of the garment. 14b is the 2nd and middle layer of the layered zone. It is a layer of latex in any form, or any other viable non breathable substance. It is enclosed between the two layers of breathable fabric, and like 14a and 14c has the vertical elongated opening, in which the seams of the two sides overlap each other by 1 to 2 inches down the center seam of the garment. 14c is the inner and 3rd layer of the layered zone. It is a layer of breathable fabric, which may be the same fabric the whole garment is made of, or any other breathable fabric, it is the layer closest to the skin. It has the same opening in the same location of the other layers for the vertical elongated opening, which consists of a layered zone that is stitched according to the seams in the undergarment. FIG. 15, is the elongated vertical opening in the genital area of the layered zone. The opening is made by the overlapping of fabric at the coming together of the two sides. There is an overlapping of fabric stitched down the middle from the top center of the garment to the dotted line. Then from the dotted line to the dotted line shown in the center of FIG. 8 it is not sewn together, but left to where the 2 sides can be pulled apart and open as in DRAWING 9 (FIG. 15b). FIG. 13 seen in the drawings is the seam. DRAWING 2 is the exploded view of FIG. 14, the layered zone, which is composed of three layers, FIG. 14 (a)(b)(c). FIG. 14a is the first and outer layer of the layered zone that is made of the breathable fabric that is used throughout the garment; it is connected to the other two layers 14(b) and 14(c), to make up the layered zone. FIG. 14(b) is the layer of latex (or other non-breathable substance/fabric), that is enclosed between the two layers of breathable fabric. FIG. 14(c) is the inner and third layer of the layered zone; it is made of a breathable fabric, and is the layer closest to the skin. These layers are formed together in halves for the front only of the garment. The halves are stitched together, shown as FIG. 13, where there is 1 to 2 inches of overlapping fabric in the center, to form the vertical overlapping of fabric opening. DRAWINGS 8 and 9 show an optional opening for the garment, FIG. 16. DRAWING 9 shows the opening(S) in an open position. DRAWING 8 shows the opening(S) in a closed position. DRAWINGS 10, 11, and 12 are the versions of a male garment. Male garments have FIG. 14 which includes (a), (b), and (c), the layered zone.

### DETAILED DESCRIPTION OF THE INVENTION

The PLEASURABLE PROTECTIVE UNDERGARMENT is a garment that seeks to address the issues of safe sex being boring, obtrusive, unreasonable, and burdensome. The idea of protection has become unimportant and optional, when it actually is very important. The PLEASURABLE PROTECTIVE UNDERGARMENT is a garment that seeks to lessen the hassle of many of the prior unaccepted methods and provide protection and pleasure in an unobtrusive and fashionable manner.

A goal of the PLEASURABLE PROTECTIVE UNDERGARMENT is to solve the problem of the prior arts, by presenting a pleasurable protective under garment that makes protection fun, acceptable, practical, sexy, convenient, easy to use, without being bulky and distracting. This garment gives sexually active people free of STD's the power to control their clear status. The garment is for infected persons, and uninfected person's not wishing to risk catching incurable diseases that are not easily detected.

The garment seeks to prevent the spreading of herpes, venereal warts, hpv, molluscom cantagosum, pubic lice (crabs), and scabies, which are spread by skin on skin contact. The protective garment should be used to help prevent skin on skin contact during sexual experiences. The garment creates a breathable, unobtrusive barrier between partners participating in sex, providing areas for opening and inserting sexual organs.

The garment is made of a breathable fabric styled similar to underwear that seeks to prohibit the transfer of sexually transmitted diseases by way of an infected person's skin. The invention in the most basic form is a dry fit fabric, shaped similar to underwear, doubly enforced in the crotch area with a layer of impermeable fabric (such as latex, vinyl, etc.) which has an area that can be opened by pulling back fabric on both sides. The invention does not have an area in which the garment is open exposing the vaginal or penile area. It however, can be opened for the insertion of the penis. This opening is accommodating for all penis sizes, and the remaining fabric encloses around the penis. If room still exists, the garment would allow for clitoral or scrotal stimulation. Additional room is dependent on how much space is already utilized.

There are both male and female garments. The invention is made differently for males and females to address the different locations of the male/female anatomy. The female garment has a long insertion area which allows for vaginal, clitoral, or vaginal penetration and clitoral stimulation, and can be comfortably worn unopened as underwear. The male garment has a long insertion area which can remain in a closed position, and worn regularly as underwear. The male garments opening area may also allow extra space for scrotal stimulation. Additional space is dependent on how much space is already utilized. Both male and female garments offer alternative styles to include an additional opening for anal usage.

### THE OBJECTIVES OF THE INVENTION

The main objectives of this invention are:
1) To create a protective barrier for the genital areas, seeking to prevent the spreading or transmitting of skin to skin contact diseases and infections during sex
2) A garment that can be worn comfortably throughout the day, as not to ruin the moment, interfere with sensations, or have to maneuver other parts. A garment that creates a go with the flow moment, and enables spontaneity, foreplay, and manual stimulation of all sexual parts.
3) To pick up protection where condoms leave off, by providing a barrier for the crotch and thigh areas, which are susceptible to contracting STD's as well.
4) Garments that can be worn easily as underwear are, providing easy access for toilet use.
5) Garments for couples in which one partner is infected with the incurable STD herpes, seeking to protect the other partner from risk of infection.
6) To provide an additional layer of protection with latex or other impermeable fabric, along with a breathable fabric, providing both comfort and protection.
7) Create a barrier between sexual partners that seeks to prevent the spread of noticeable and unnoticeable skin on skin contact infections.
8) To create a garment that when worn in addition to a condom seeks to provide total protection.

1. An undergarment in the form of underpants, in that they cover the: waist, lower abdomen, crotch, buttocks, and upper to mid-thigh area. Said undergarment is
A. composed of 3 layers
1. the whole garment is made of a breathable fabric
2. a (1) layer of latex is enclosed between the (2) layers of breathable fabric (an outer and inner), specifically in the crotch area
B. has a vertical elongated opening in the center of the crotch area of the garment
1. optional garments have 2 openings
C. has an overlapping of fabric that leads to the opening(s) of the garment
D. Said garment is not bound by fabric or style

2. (canceled)

3. (canceled)

4. (canceled)
